(19) United States
(12) Patent Application Publication
(10) Pub. No.: US 2008/0182251 A1
(43) Pub. Date: Jul. 31, 2008

(54) Ultra Low Strength Electric Field Network-Mediated Ex Vivo Gene, Protein and Drug Delivery in Cells

(76) Inventors: Luyi Sen, Stevenson Ranch, CA (US); Guangen Cui, Stevenson Ranch, CA (US)

Correspondence Address: MYERS DAWES ANDRAS & SHERMAN, LLP, 19900 MACARTHUR BLVD., SUITE 1150, IRVINE, CA, 92612, US

(21) Appl. No.: 11/909,074

(22) PCT Filed: Mar. 16, 2006
(86) PCT No.: PCT/US06/11355

§ 371 Date: Sep. 18, 2007

Related U.S. Application Data
(60) Provisional application No. 60/663,562, filed on Mar. 19, 2005.

(51) Int. Cl. C12N 15/01; C12M 1/42; C12Q 1/68; C12Q 1/02

(52) U.S. Cl. 435/6; 435/446; 435/285.2; 435/29

(57) ABSTRACT

Ex vivo gene, protein or drug delivery to macroscopic quantities of various types of cells, cell clusters, or tissues using ultra low strength LSEFN strategies is disclosed in which the bioengineered cells and tissues are then systemically transfused, delivered or implanted into the various organs or tissue for the treatment of diseases. An LSEFN chamber is used which is shaped and sized to intimately contain the cells, cell clusters, or tissues in a transfusion chamber between opposing membrane encapsulated electrode arrays across which LSEFN pulses are applied.

The present application is related to U.S. Provisional Patent Application Ser. No. 60/663,562, filed on Mar. 19, 2005, which is incorporated herein by reference and to which priority is claimed pursuant to 35 USC 119.

### BACKGROUND OF THE INVENTION

1. Field of the Invention

The invention is in the field of methodologies to facilitate the ex vivo gene, protein or drug delivery in large quantity to various types of cells or cell clusters, such as islets, or various of tissues using an ultra low strength electric field network.

2. Description of the Prior Art

Electroporation is a technique involving the application of short duration, high intensity electric field pulses to cells or tissue. The electrical stimulus causes membrane destabilization and the subsequent formation of nanometer-sized pores. In this permeabilized state, the membrane can allow passage of DNA, enzymes, antibodies and other macromolecules into the cell. Electroporation holds potential not only in gene therapy, but also in other areas such as transdermal drug delivery and chemotherapy.

Since the early 1980s, electroporation has been used as a research tool for introducing DNA, RNA, proteins, other macromolecules, liposomes, latex beads, or whole virus particles into living cells. Electroporation efficiently introduces foreign genes into living cells, but the use of this technique had been restricted to the small quantity of suspensions of cell lines and primary cultures for basic research only, since the electric pulse are administered in a cuvette with a pair of needle type electrodes. No system has been established for the ex vivo low strength electroporation-mediated gene, protein or drug delivery to a large quantity of cells or a cell cluster, such as islet for therapeutic use.

Electroporation is commonly used for in vitro gene transfection, but limited work has been reported for in vivo gene transfer using a pair of needle or plate form electrodes in tumor, liver, myocardium in rodents. Most recently, an electroporation catheter has been used for delivery heparin to the rabbit arterial wall, and significantly increased the drug delivery efficiency. Most recently, we invented the systems for low strength electroporation-mediated in vivo gene, protein and drug delivery in organ and tissue of large animal and human (See U.S. Pat. No. 6,593,130 (2003) incorporated herein by reference. In that invention we also described a device for low strength electroporation-mediated ex vivo gene, protein and drug delivery in vessel of large animal and human.

No system has been described for the ex vivo low strength electroporation-mediated gene, protein or drug deliver into tissue or bioengineered tissue culture for therapeutic use. On the other hand, electric pulses with high electric field intensity can cause permanent cell membrane breakdown (cell lysis). According the best of knowledge now available, the voltage applied to any kind cells, whole embryo or embryonic heart in the cuvette setting must be as high as 200-1500 V/cm, and to any in vivo tissue using needle or plate form electrodes must be as high as 100-200 V/cm. Injury in such cases is a major concern, although it has never been well characterized.

### BRIEF SUMMARY OF THE INVENTION

One object of the invention is to establish the concept and applicable methodology for facilitate the ex vivo gene, protein or drug delivery to large quantities of various types of cells or cell clusters, such as islet, or various types of tissues using ultra low strength electroporation, which is defined in this specification as low strength electric field networking (LSEFN) strategies. The mechanism and nature of the bioelectric application in the present invention is only now being appreciated as being qualitatively different than prior art electroporation. Hence, to refer to the present bioelectric application as electroporation is misleading and inaccurate. Thus, hereinafter in this specification and in the medical field the present bioelectric application is referred to as low strength electric field networking (LSEFN). These bioengineered cells and tissues can then be transfused systemically, delivered or implanted into the various organs or tissue for the treatment of diseases.

The invention includes two components: 1) ex vivo gene, protein and drug delivery into the various of isolated cells mediated by the ultra low strength electric field; 2) ex vivo gene protein and drug delivery into cell clusters, such as islet, or whole embryos by the ultra low strength electric field; and 3) ex vivo gene, protein and drug delivery into the various types of cultured and bioengineered tissue using an ultra low strength electric field.

An LSEFN chamber is used which is shaped and sized to intimately contain the cells, cell clusters, or tissues in a transfusion chamber between opposing gas permeable membrane encapsulated electrode arrays across which low voltage LSEFN pulses are applied. The gas or gases introduced into the culture fluid through the gas permeable membrane can be chosen to optimize the specific metabolism and health required by the cell, cell clusters, or tissues. The high percentage of cell death, which is typical of prior art electroporation, is minimized or even avoided in the present application by the synergistic combination of low strength electric field network and optimal culture and gas environment for the cell, cell clusters, or tissues.

The illustrated invention is thus characterized by and has the advantages of: low voltage electro-permeabilization which results in less damage to the cell; dynamic electro-permeabilization, i.e. cells are moving in a static field, and rotating in a constant rate of buffer flow, therefore a constant temperature is maintained and heat damage to the cells is avoided thus allowing a long term LSEFN treatment as compared to the prior art; use of an electric array of very small electrodes to minimize heat and to use diffusing electric fields for providing a more nearly uniform average LSEFN exposure and transfusion into the cells; and a non-cuvette system which uses long exposure cell in a compact chamber to transfuse a large number of cells and to reintroduce them at a single time for large batch processing.

The invention is thus defined in its illustrated embodiment as a method of delivery of gene, protein or drug materials to macroscopic quantities of cells, cell clusters, or tissues comprising the steps of applying ex vivo LSFEN electric field to the cells, cell clusters, or tissues with an averaged field strength and an averaged electrical polarization of the LSEFN electric field; and systemically transfusing the gene, protein or drug materials into the cells, cell clusters, or tissues during LSEFN.

The method further comprises delivering in vivo the transfused cells, cell clusters, or tissues into organs or tissue.

The method further comprises the step of flowing a culture fluid to bathe the cells, cell clusters, or tissues during application of ex vivo an LSEFN electric field and during systemically transfusing the gene, protein or drug materials. The fluid may be used to culture the cells, cell clusters, or tissues. The flowing culture fluid easily mixes with the drug, protein or gene and increases the chance of the drug, protein or gene interaction with the cell membrane.

The step of applying ex vivo LSEFN electric field to the cells, cell clusters, or tissues comprises applying a pulsed DC electrical field with a predetermined burst repetition rate, each burst being separated by a predetermined rest period. The method step of applying ex vivo an LSEFN electric field to the cells, cell clusters, or tissues comprises applying an LSEFN electric field of less than 100 v/cm and preferably at approximately 10-1 V/cm or less. Regardless, of the numerically determined value of the LSEFN electric field, it is chosen at a magnitude which does not cause dielectric heating and biological damage to the cells, cell clusters, or tissues.

A flowing fluid maintains the temperature of the fluid substantially constant to avoid heat damage to the cells, cell clusters, or tissues in the LSEFN electric field.

The illustrated method of applying ex vivo an LSEFN electric field comprises the steps of disposing the cells, cell clusters, or tissues to the LSEFN electric field between at least one pair of electrodes across which the electric field is imposed. The electrodes are arranged and configured to provide a fringing field between them and being separated by a distance such that the cells, cell clusters, or tissues are primarily exposed to the fringing field so that the cells, cell clusters, or tissues are exposed to the averaged field strength and the averaged electrical polarization of the LSEFN electric field. An array of a pair of electrodes providing a positive and negative grid may be employed or a plurality of subarrays of various electrode elements employed in varied geometric arrangements.

The cells, cell clusters, or tissues are disposed into a chamber containing the cells, cell clusters, or tissues. The electrode arrays are disposed on or in the walls of the chamber, which walls intimately conform to the cells, cell clusters, or tissues subject to LSEFN, thereby providing the averaged field strength and the averaged electrical polarization of the LSEFN electric field. The LSEFN electric field is generated or applied using multiple arrays of a plurality of small electrodes to generate a pixilated fringing electric field. The size of the electrodes are chosen relative to the size of the cells, cell clusters, or tissues to provide an effective averaged field strength and the averaged electrical polarization of the LSEFN electric field at that scale.

The averaging of the field strength and electrical polarization of the LSEFN electric field is provided in one embodiment by flowing a fluid to bathe the cells, cell clusters, or tissues comprises moving the cells, cell clusters, or tissues in the LSEFN electric field, which translates and rotates the cells, cell clusters, or tissues in the LSEFN electric field. The rotation may be chaotic and comprise random tumbling of the cells, cell clusters, or tissues in the LSEFN electric field. The flow keeps the cells and cell clusters rolling in the electric field and receiving homogenously distributed LSEFN while the cell, cell clusters and tissue maintained in a sterile and nutritional cell and tissue culture environment.

The method is easily modified to mass production or batch production of a large number of cells, cell clusters, or tissues during a single exposure time interval over an extended exposure path along which the cells, cell clusters, or tissues are moved, whereby mass production of mediated cells, cell clusters, or tissues are produced.

The systemic transfusing of the gene, protein or drug materials into the cells, cell clusters, or tissues during LSEFN is microscopically observed, examined and tested with respect to the alterations of cells, cell clusters or tissues microscopically during LSEFN with respect to ability of the cells, cell clusters or tissues for later gene, protein or drug delivery.

The invention further includes apparatus for delivery of gene, protein or drug materials into macroscopic quantities of cells, cell clusters, or tissues according to any one of the foregoing methodologies.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1a is a top plan view of a diagram of a chamber in which low voltage LSEFN may be practiced.

FIG. 1b is diagrammatic cross sectional view of the chamber of FIG. 1a.

FIG. 2a is an exploded perspective view of the two permeable membranes and the electrode array sandwiched between them to form a flexible encapsulated assembly as seen to the right in the drawing.

FIG. 2b is a partially disassembled perspective view of the two opposing encapsulated assemblies with the side and end frames of the embodiment of FIGS. 1a and 1b.

FIG. 2c is a top plan view of the assembled embodiment of FIG. 2b.

FIG. 3a is a side plan view of a diagram of a second embodiment of the chamber provided as a flexible cylindrical tube in which low voltage LSEFN may be practiced.

FIG. 3b is a perpendicular cross-sectional view taken through section lines 3b-3b of FIG. 3a.

FIG. 4 is diagrammatic perspective view of the chamber of FIGS. 3a and 3b configured to form a helical assembly.

FIG. 5 is a top plan view of a diagram of a third embodiment of the chamber in which low voltage LSEFN may be practiced.

FIG. 6 is diagrammatic longitudinal cross sectional view of the chamber of FIG. 5.

FIG. 7 is a wave timing diagram showing a typical pulse sequence in the low voltage LSEFN process of the invention.

FIG. 8 is a diagrammatic depiction of a laboratory setup wherein the invention may be practiced with real time microscopic observation of the LSEFN.

FIGS. 9a and 9b are diagrams showing the construction of human plasmids IL-10 cDNA used for cationic liposome mediated HIL-10 gene transfer and adenovirus-mediated gene transfer respectively. FIG. 9c is a bar graph showing the percentage of gene transfer.

FIG. 10a is a photograph of a superimposed gel showing transgene expression. FIG. 10b is a bar graph of the transgene/GAPDH ratio verses electrical field strength for the LSEFN in volts/cm.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

In the illustrated embodiment of the invention what is disclosed is a method and apparatus for electro-permeabilization of large quantities of isolated cells and cultured tissues ex vivo for use in gene, protein, and drug targeting using ultra low electric field strengths, short pulse duration, and long burst pulse duration. To enable the application of ultra low strength electric fields for highly efficient gene, protein and drug delivery in isolated cells, cell clusters and cultured tissues, we also designed three different embodiments of a novel gene, protein and drug delivery system. However, it must be expressly understood that the illustrated embodiments do not exhaust the scope of embodiments which are intended to fall within the reach of the claims.

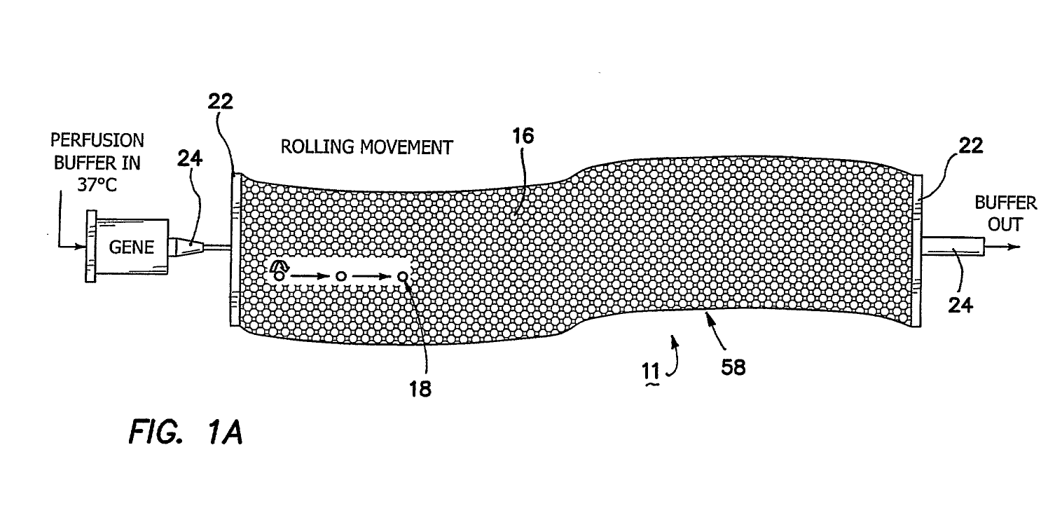

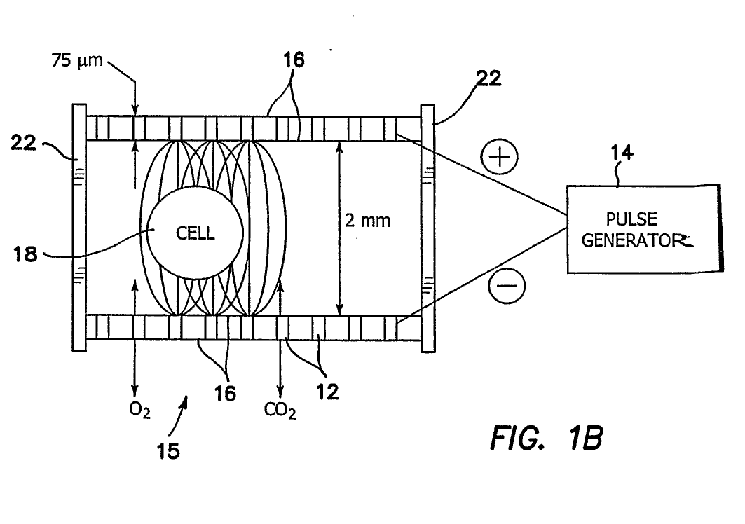

These embodiments include an apparatus 11 for applying ultra low strength electric-fields to provide for mediated ex vivo gene, protein and drug delivery to isolated cells. The apparatus 11 is diagrammatically shown in FIG. 1a in top plan view, in side cross sectional view in FIG. 1b, in partially disassembled perspective view in FIG. 2b and in assembled top plan view in FIG. 2c. In this system, an electrode array 10 is sealed between two opposing, flexible gas permeable and transparent membranes 16a and 16b, collectively denoted by reference numeral 16, which are approximately 70 μm thick. The membranes 16 need not be flexible, but the flexibility allows the apparatus 11 to be folded or shaped to a more compact volume without loss of its two dimensional extent. Opposing membranes 16a and 16b are spaced apart by a distance sufficient to allow tumbling of the cells flowing in a buffer between them, but small enough to provide intimate exposure to an electric field at ultra low voltages as discussed below which is employed to provide LSEFN of the cells. The membranes 16 are combined with an opposing pair of flexible sides 17 to form a sealed rectangular frame 15. Membranes 16 are composed of polystyrene, which is selectively gas permeable to O₂ and CO₂. In the illustrated embodiment each membrane is about 75 μm thick, which allows for efficient O₂ and CO₂ exchange between both membranes, but separates the cellular environment from ambient atmosphere. This allows for optimal oxygenation of the growing cells and provides a balanced pH medium.

Membranes 16 are fabricated to encapsulate the array of electrodes 12 as best shown in exploded view in FIG. 2a, wherein electrode sheet 12 is sandwiched between membrane sheets 16a and 16b, which are then bonded together to form the encapsulated electrode assembly 13.

Membranes 16 are preferably transparent to allow microscopic observation of cells 18 and is gas permeable to allow and to facilitate culturing of cells 18 in a chamber 20 defined between opposing encapsulated electrode assemblies 13. Using two opposing electrode-membranes assemblies 13, a sterile and sealed chamber 20 is formed in a rectangular plastic frame 15 comprised of end plates 22, flexible top and bottom membrane panels 19 and flexible side panels 21 as best shown in FIG. 2b. The panels 19 and 21 of frame 17 are flexible to allow for the apparatus 11 to be configured into a compact three-dimensional shape while allowing an extended linear length of chamber 20 extending between the two opposing end plates 22. Two perfusion needles 24 are fixed on and extend through the opposite end plates 22 of chamber 20 to allow fluid injection and discharge and to maintain a buffer flow through chamber 20.

In the preferred embodiment of the method of the invention, a large quantity of isolated cells 18 is injected into the culture chamber 20 through one perfusion needle 24, which cells fill chamber 20. The chamber capacity is between approximately 10-1000 ml or more, but can be varied as may be appropriate in end application. A culture medium or buffer including selected gene, protein or drug material or materials is continuously perfused through the chamber 20 with a rate at approximately 10 ml/hour to keep the cells 18 moving and unadhered to the membranes 16a, 16b and is maintained at a temperature of 37° C. It is of course to be understood that the nature of the buffer, its temperature, flow rate and other culture parameters can be varied according to the number, type and nature of LSEFN, cells, genes, proteins or drug materials at hand according to conventional culture principles.

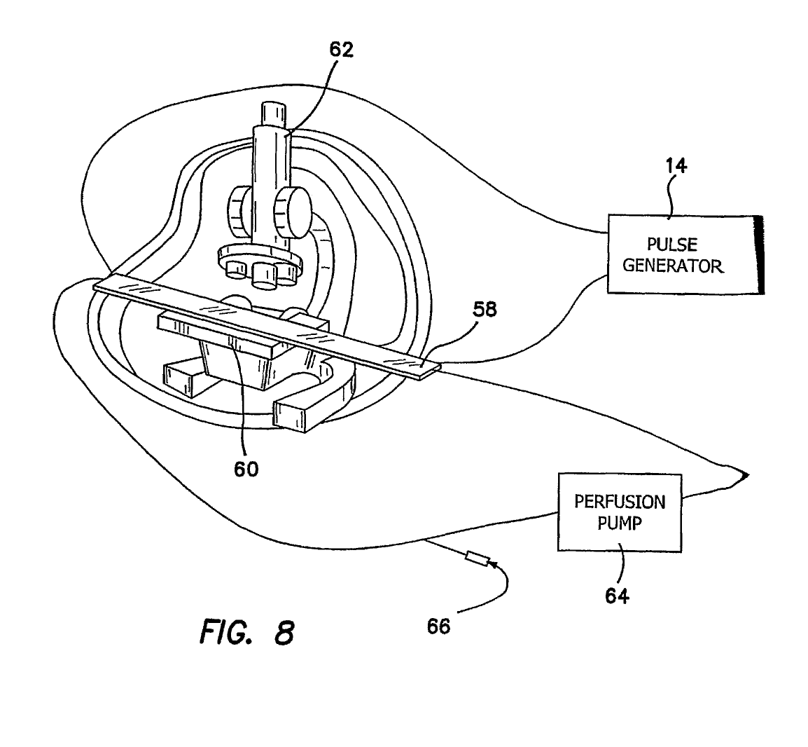

A simple laboratory setup in which the invention may be practiced is schematically shown in FIG. 8. A strip chamber 58 such as shown and described in connection with FIGS. 1a and 1b is connected to pulse generator 14 and mounted on a microscope stage 60 of microscope 62 to permit microscopic observation during the LSEFN process. In a production unit microscopic observation might not be required or could be provided by means that accommodate the nature of strip chamber 58 as it may be embodied. Strip chamber 58 may be manually movable or may be movable by a motorized stage or mechanism (not shown) controlled by a joystick or other means to allow for selective observation of any region to determine motion of the buffer, the cells and materials in the buffer and their electroporation state. It is of course contemplated that strip chamber 58 may be stationary and the microscope 62 moved instead. Strip chamber 58 is coupled to a perfusion pump 64 which keeps the buffer flowing and contents thereof moving through or in chamber 58. The buffer can be loaded by injection of cells, genes, proteins, drugs or culture buffer solutions through a valved port 66.

Optimized electric pulses are applied from pulse generator 14 to both sides of the electrode array 12, which are planar arrays or grids as described above in FIGS. 1a and 1b, with one array 12 being connected to a positive DC voltage and the other to a negative DC voltage. The detail of the array 12 may be a screen or other configuration chosen according electromagnetic design principles and the object of providing a diffuse or fringing electric field in strip chamber 58. The optimization of the pulse form may be determined from conventional principles or from trial and error. The LSEFN pulses applied to array 12 and across the buffer may assume any pulse profile, repetition rate and pulse shape which is now known or later devised. The pulse profiles used are generally conventional and well known within conventional methodologies and are adjusted for each particular application at hand. For example, any of the pulse profiles disclosed in U.S. Pat. No. 6,593,130 (2003) may be employed and as further may be modified to be consistent with or adapted to the teachings of the present invention according to well understood biophysical principles.

The distance separating the two opposing sides of the electrode array 12 is approximately 5 mm in the illustrated embodiment. The strength of the electric field applied across the chamber 20 is approximately 5 volts/cm during the perfusion. This is much lower than that in the conventional cuvette setting of 200-1500 volts/cm and hence is defined for the purposes of this specification and its claims as an ultra low electrical field strength. The treatment need last only about 20-60 minutes. Chamber 20, however, can also be used for long-term culture. The cells 18 can be observed under a microscope while still in chamber 20 which can be placed in an incubator for long time culturing. The treatment can be repeated if desired.

FIG. 1b diagrammatically illustrates that cell 18 is exposed in chamber 20 to a fringing or diffuse electrical field. Furthermore, as the cells 18 flow with the buffer down the longitudinal axis of chamber 20 they tend to tumble or rotate further averaging both the magnitude and polarizations of electrical field to which the cell membrane is exposed. The result is a more uniformly electroporated and transfused cell or target than would be the case in a static buffer.

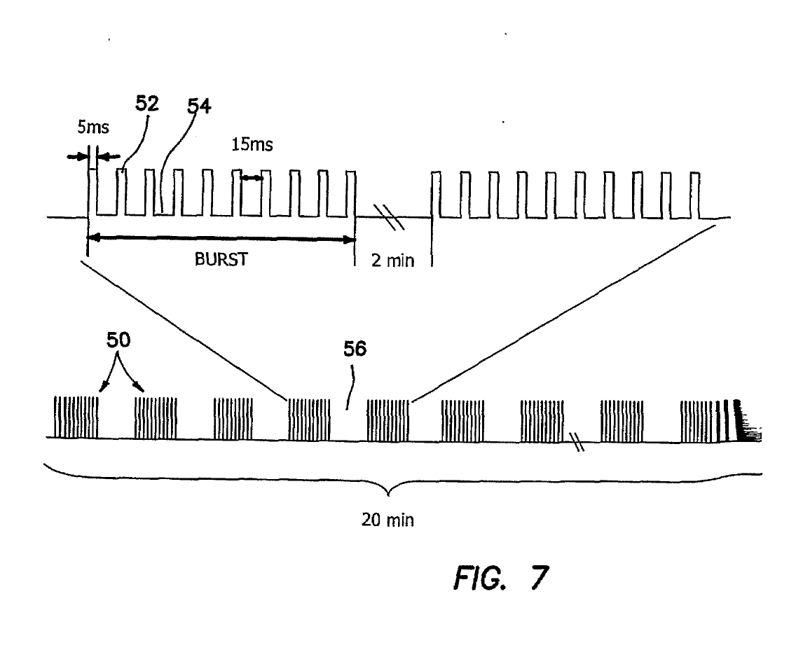

A typical pulse profile is shown in more detail in FIG. 7 as comprised of a plurality of pulse groups 50 spaced over approximately a 20 minute total exposure period. The period of exposure can be varied in a manner consistent with the teaching of the invention over longer or shorter total exposure times. A rest or null period 56 of approximately 2 minutes, where there is no or substantially no effective electric field exposure, is provided between pulse groups 50. Again the rest or null period 56 can be longer or shorter according to the application at hand. The pulse group 50 is shown in FIG. 7 as illustratively comprised of a plurality of 5 ms pulses 52, each separated by 15 ms zero or null field intervals 54. The duration of the burst of group 50 is variable according the teachings of the invention and may be optimized empirically in each case.

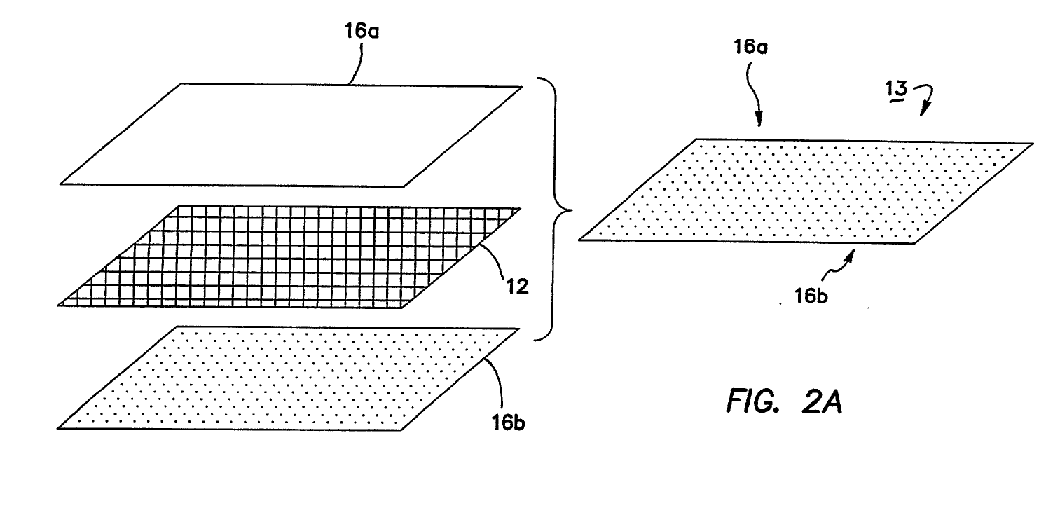

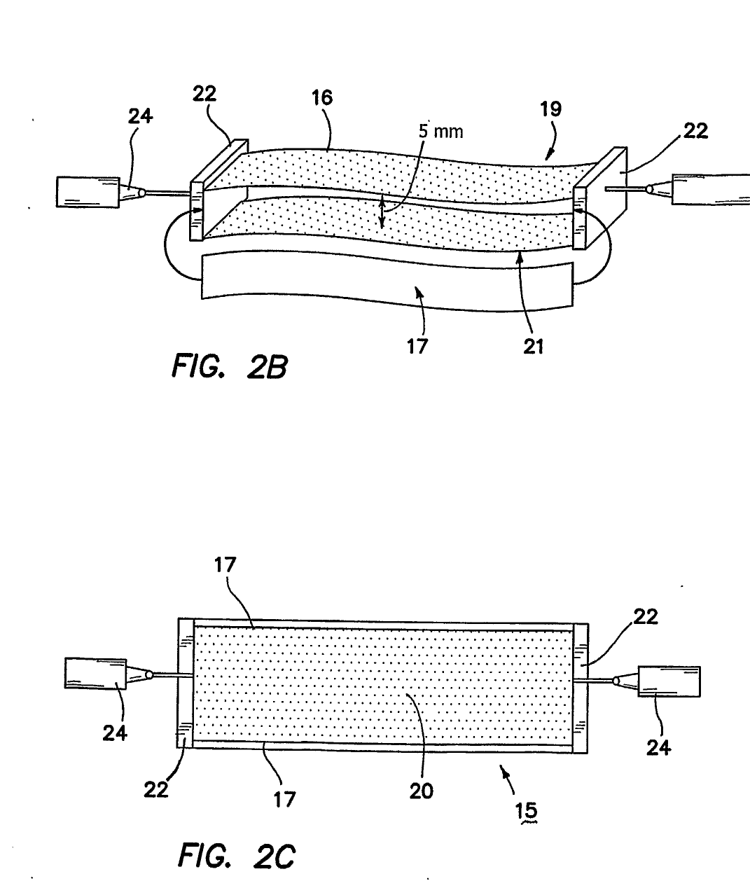

In the second embodiment of FIGS. 3 and 4 we provide an apparatus 11 for ultra low strength electric-field mediated ex vivo gene, protein and drug delivery in cells, and clusters, such as islets. A cylindrically shaped or tubular culture chamber 26 is provided with an electrode array 12 similar to that shown in FIGS. 1a, and 2b and is used for applying the electric field from generator 14 to the cell clusters 28. As shown best in the cross-section view of FIG. 3b, chamber 26 is provided with encapsulated electrodes 12 between concentric membranes 16, the electrodes 12b being connected to the negative terminal 40 and electrodes 12a being connected to the positive terminal 42. Electrodes 12a and 12b may be provided in any geometric arrangement desired, but the preferred embodiment is shown in FIG. 3b where the negative electrodes 12b and positive electrodes 12a are geometrically alternated to maximize the fringing field which extends into chamber 26 and hence to cluster 28. The number of electrodes is shown diagrammatically in FIG. 3b and it must be understood that the number of electrodes and their shape are matters of design that can be varied in many ways in a manner consistent with the teachings of the invention.

FIG. 4 shows that the cylindrical chamber 26, which is unrolled or shown in a linear shape in FIG. 3a, can be helically coiled to form a more three-dimensionally compact system. In this chamber 26 as best seen in the perpendicular cross-sectional view of FIG. 3b, the electrode arrays 12 are closer to the cell clusters 28 than in the first embodiment because the chamber dimensions more nearly approximate the size of the cell clusters themselves. Thus, the LSEFN voltage can be further reduced. For example of field of about 1 V/cm can be employed across chamber 26, which is again included within the definition of ultra low field strength.

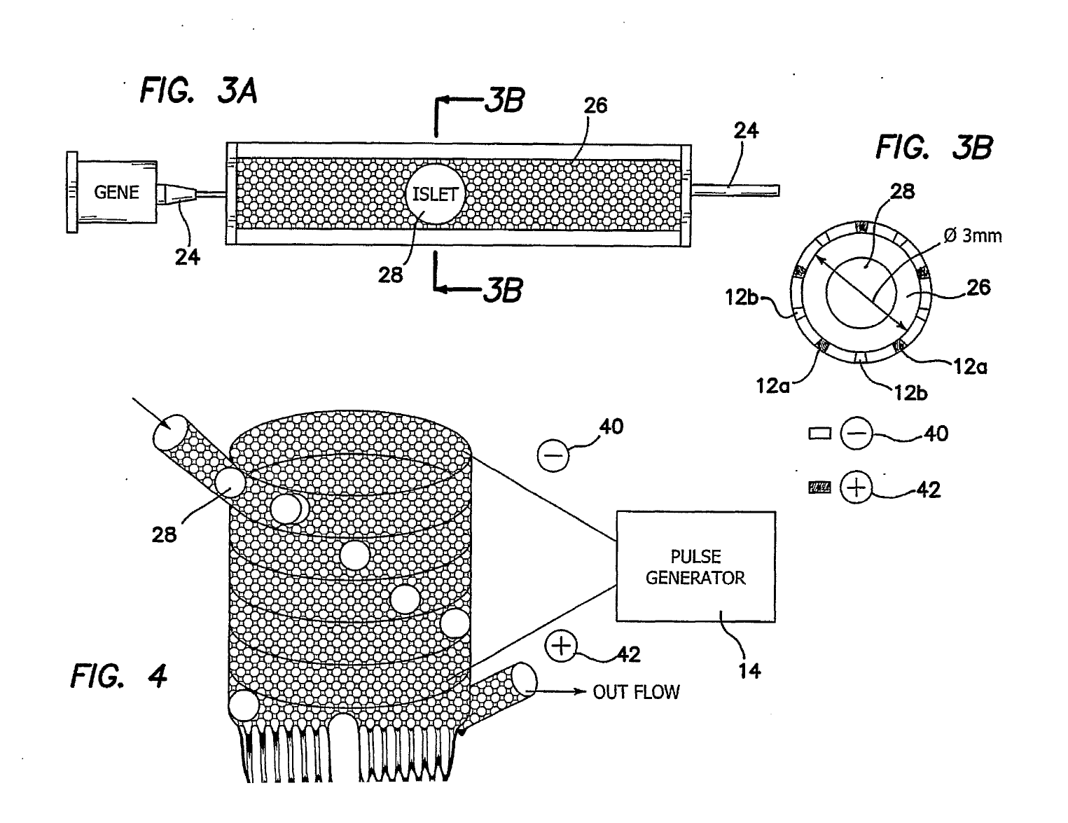

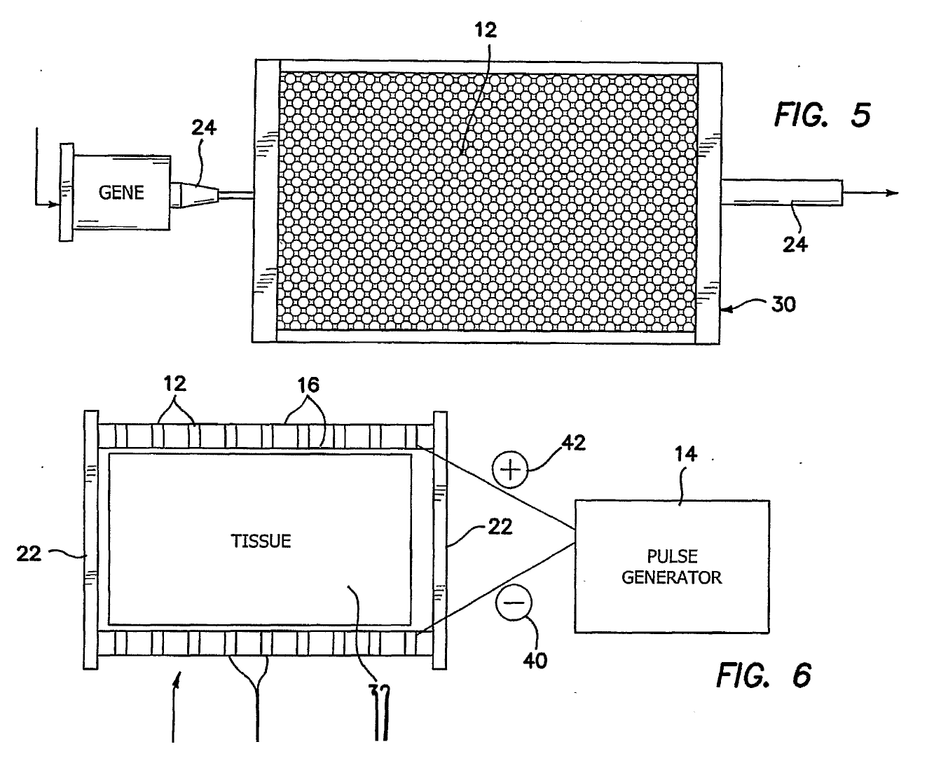

In the third embodiment of FIGS. 5 and 6 we provide another apparatus 11 for ultra low strength electric-field mediated ex vivo gene, protein and drug delivery in tissue 32. In this embodiment, the chamber size and shape can be modified to match the various shapes of cultured tissue, or bioengineered scaffolds 32. Two opposing electrode arrays 12 in sealed membranes 16 are provided on the both side of the culture tissue 32 in chamber 30 in a manner similar to FIGS. 1-4. However, the size and shape of frame 22 and the chamber 30 defined within it is customized to the particular shape of the tissue being treated. For example, a section of skin graft tissue or cornea may be the treated tissue in which case frame 22 and the chamber 30 will be contoured to match the section of skin graft tissue, so that low field LSEFN can be effectively performed on the section during perfusion.

Consider now how the invention is used to provide gene, protein and drug therapy for isolated cells. Any biological cells, such as lymphocytes, monocytes, bone marrows, myoblaste, stem cells, etc, can be isolated from the human body. Then ex vivo delivery of gene, protein or drugs into these cells using ultra low strength electric field is then systemically or locally infused or injected in the patients for therapeutic purposes. For example, in cancer therapy, the monocytes can be isolated from a patient and ex vivo delivery the CXCR3 gene into these cells, then injected into the lung for immunotherapy for lung cancer. In stem cell transplantation, some anti-apoptosis gene can be transfected into the stem cell before it been transplanted into the targeted organ.

Similarly consider how the invention is used to provide gene, protein and drug therapy for a cell cluster. For example in islet cell cluster transplantation, the allogenic or exogenic islet cell cluster can be ex vivo delivered with the immuno-suppressive genes to prevent rejection.

The invention is also used to provide gene, protein and drug therapy for cultured and engineered tissue. For example: in tissue engineer, the PET reporter gene can be delivered into the tissue during culture.

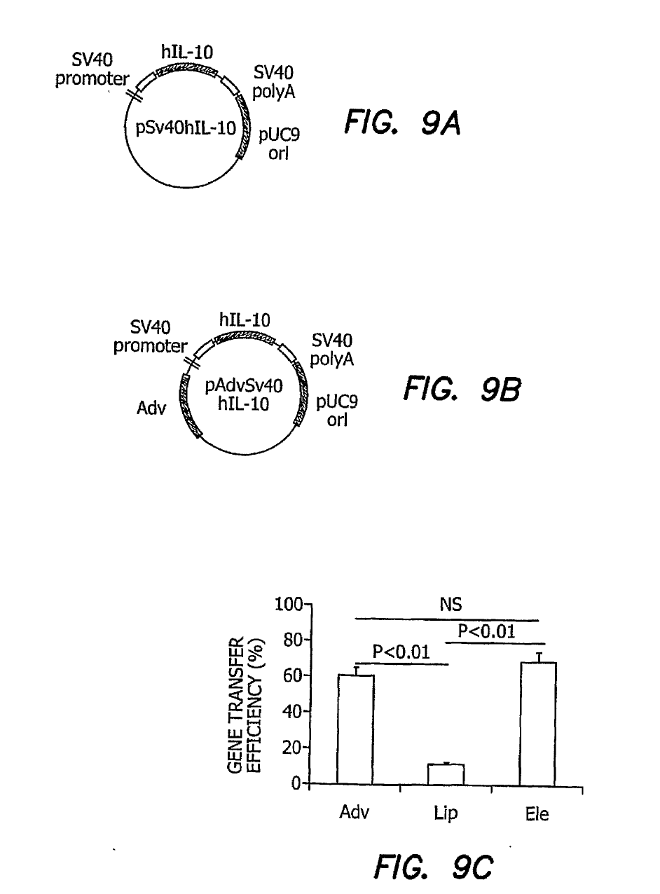

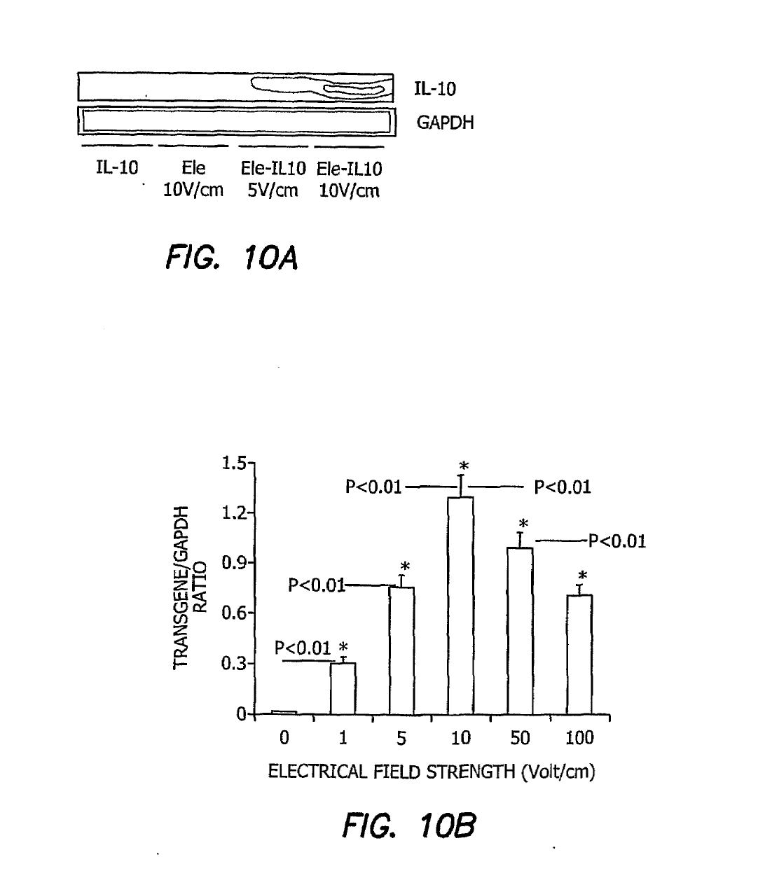

FIGS. 9a, 9b, 9c, 10a and 10b graphically illustrate the results of a preliminary usage of invention. FIG. 9a is a diagram of the structure of a human plasmid. The human plasmid IL-10 cDNA is used for cationic liposome (Gap:DLRIE)-mediated hIL-10 gene transfer and ultra low strength LSEFN-mediated hIL-10 gene transfer. FIG. 9b illustrates the construction of Adenovirus-human plasmid IL-10 cDNA used for adenovirus-mediated gene transfer. FIG. 9c is a bar graph that illustrates the efficiency of in vitro human IL-10 gene transfer in human peripheral lymphocytes mediated by adenovirus (Adv, n=5), liposome (Lip, n=5) or ultra low strength (10 volt/cm) LSEFN (Ele, n=5). To determine the gene transfer efficiency, antisense and sense digoxygenin-labeled riboprobes (Boehringer Mannheim) of hIL-10 mRNA were synthesized and used for in situ hybridization on paraffin section as described previously. The gene transfer efficiency was determined as the percentage of blue-stained positive cells in total lymphocytes counted in 10 high power microscopic fields (magnification, ×400) per section. The efficiency of in vitro gene transfer mediated by ultra low strength LSEFN was five fold higher than liposome-mediated gene transfer and slightly higher than adenovirus-mediated gene transfer

FIG. 10a is a superimposed gel illustration which shows the representative data of human IL-10 transgene expression detected by quantitative competitive RT-PCR analysis. Transgene expression was not detected in lymphocytes transfected by human-IL-10 vector alone without LSEFN (lane 1) or treated with 10 volt/cm LSEFN without perfusion with vector (lane 2). The transgene expression was significantly higher in lymphocytes treated with human IL-10 gene and 10 volt/cm LSEFN (lane 4) compared with that treated with 5 volt/cm LSEFN (lane 3).

FIG. 10b is a bar graph in which electrical field strength effect on the ultra low strength LSEFN-mediated on vitro human IL-10 transgene expression ratios in human lymphocytes is plotted. Transgene expression is detected by quantitative competitive RT-PCR analysis. Data is represented as the ratio of IL-10/GAPDH RT-cDNA level. n is equal 3-5 at each data point. The transgene expression level was highest when a 10 V/cm electric field strength was applied. P was less than 0.01 when compared with that in control (0 volts/cm).

The foregoing examples by no means exhausts the very large number of applications in which low field LSEFN can be used to deliver ex vivo gene, protein and drug therapy, which then can be used for human or animal intervention, therapy and disease prevention. The invention has a number of advantages or improvements over existing practices. The invention opens a new era in the gene, protein and drug targeting for the prevention and treatment of large animal and human disease. Prior to the invention there was no existing technique that was applicable for human use. The ultra low voltage LSEFN concept and technique disclosed above gives us a powerful tool for gene transfer without the viral vectors. It has subsequently become apparent that the use of viral vectors may not always be the ideal means of delivery due to the additional genetic material in the virus and its self-replicating quality.

Many alterations and modifications may be made by those having ordinary skill in the art without departing from the spirit and scope of the invention. For example,

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. A method of delivery of gene, protein or drug materials to macroscopic quantities of cells, cell clusters, or tissues comprising:
applying ex vivo a low strength electric field networking (LSEFN) to the cells, cell clusters, or tissues with an averaged field strength and an averaged electrical polarization of the LSEFN electric field; and
systemically transfusing the gene, protein or drug materials into the cells, cell clusters, or tissues during LSEFN.

2. The method of claim 1 further comprising flowing a fluid to bathe the cells, cell clusters, or tissues during application of ex vivo an LSEFN electric field and during systemically transfusing the gene, protein or drug materials.

3. The method of claim 2 further comprising flowing the fluid to culture the cells, cell clusters, or tissues.

4. The method of claim 1 further comprising flowing the fluid to culture the cells, cell clusters, or tissues.

5. The method of claim 1 further comprising delivering in vivo the transfused cells, cell clusters, or tissues into organs or tissue.

6. The method of claim 1 where applying ex vivo an LSEFN electric field to the cells, cell clusters, or tissues comprises applying a pulsed DC electrical field with a predetermined burst repetition rate, each burst being separated by a predetermined rest period.

7. The method of claim 1 where applying ex vivo an LSEFN electric field to the cells, cell clusters, or tissues comprises applying an LSEFN electric field of less than 100 v/cm.

8. The method of claim 1 where applying ex vivo an LSEFN electric field to the cells, cell clusters, or tissues comprises applying an LSEFN electric field of approximately 10 v/cm or less.

9. The method of claim 1 where applying ex vivo an LSEFN electric field to the cells, cell clusters, or tissues comprises applying an LSEFN electric field of approximately 1 v/cm or less.

10. The method of claim 1 where applying ex vivo an LSEFN electric field to the cells, cell clusters, or tissues comprises applying an LSEFN electric field of less than a determined value which causes dielectric heating and biological damage to the cells, cell clusters, or tissues.

11. The method of claim 1 where applying ex vivo an LSEFN electric field to the cells, cell clusters, or tissues comprises disposing the cells, cell clusters, or tissues to the LSEFN electric field between at least one pair of electrodes across which the electric field is imposed, the electrodes being arranged and configured to provide a fringing field between them and being separated by a distance such that the cells, cell clusters, or tissues are primarily exposed to the fringing field so that the cells, cell clusters, or tissues are exposed to the averaged field strength and the averaged electrical polarization of the LSEFN electric field.

12. The method of claim 1 where applying ex vivo an LSEFN electric field to the cells, cell clusters, or tissues comprises containing the cells, cell clusters, or tissues in a chamber with walls in or on which electrode arrays are disposed which generate the LSEFN electric field and which walls intimately conform to the cells, cell clusters, or tissues subject to LSEFN, thereby providing the averaged field strength and the averaged electrical polarization of the LSEFN electric field.

13. The method of claim 2 where flowing a fluid to bathe the cells, cell clusters, or tissues comprises moving the cells, cell clusters, or tissues in the LSEFN electric field.

14. The method of claim 13 where moving the cells, cell clusters, or tissues in the LSEFN electric field comprises rotating the cells, cell clusters, or tissues in the LSEFN electric field.

15. The method of claim 14 where rotating the cells, cell clusters, or tissues in the LSEFN electric field comprises tumbling the cells, cell clusters, or tissues in the LSEFN electric field.

16. The method of claim 2 where flowing a fluid to bathe the cells, cell clusters, or tissues further comprises maintaining a temperature of the fluid substantially constant to avoid heat damage to the cells, cell clusters, or tissues in the LSEFN electric field.

17. The method of claim 1 where applying ex vivo an LSEFN electric field to the cells, cell clusters, or tissues with an averaged field strength and an averaged electrical polarization of the LSEFN electric field comprises applying the LSEFN electric field using multiple arrays of a plurality of small electrodes to generate a pixilated fringing electric field.

18. The method of claim 1 where applying the LSEFN electric field to the cells, cell clusters, or tissues comprises applying the LSEFN electric field and systemically transfusing the gene, protein or drug materials to a large number of cells, cell clusters, or tissues in a batch during a single exposure time interval over an extended exposure path along which the cells, cell clusters, or tissues are moved, whereby mass production of mediated cells, cell clusters, or tissues are produced.

19. A method of delivery of gene, protein or drug materials to macroscopic quantities of cells, cell clusters, or tissues comprising:
applying ex vivo a dynamic LSEFN electric field to the cells, cell clusters, or tissues while contained in a gas permeable tissue culture chamber, which intimately conformed to the cells, cell clusters or tissues; and
microscopically observing the systemic transfusing of the gene, protein or drug materials into the cells, cell clusters, or tissues during LSEFN.

20. A method of delivery of gene, protein or drug materials to macroscopic quantities of cells, cell clusters, or tissues comprising:
applying in vitro a dynamic LSEFN electric field to the cells, cell clusters, or tissues while contained in a gas permeable tissue culture chamber which intimately conformed to the cells, cell clusters or tissues;
systemically transfusing the gene, protein or drug materials into the cells, cell clusters, or tissues during LSEFN; and
observing, examining or testing the alterations of cells, cell clusters or tissues microscopically during LSEFN with respect to ability of the cells, cell clusters or tissues for later gene, protein or drug delivery.

21. An apparatus for delivery of gene, protein or drug materials into macroscopic quantities of cells, cell clusters, or tissues comprising:
a source of LSEFN electric field presenting an averaged field strength and an averaged electrical polarization to an exposure volume; and
a systemic transfusing source of the gene, protein or drug materials into the cells, cell clusters, or tissues during LSEFN.

22. The apparatus of claim 21 further comprising a bath of flowing fluid in which the cells, cell clusters, or tissues are disposed during application of ex vivo LSEFN electric field and during systemically transfusing the gene, protein or drug materials.

23. The apparatus of claim 22 where the bath is a culture bath.

24. The apparatus of claim 21 where the source of the LSEFN electric field comprises a pulsed DC electrical field source with a predetermined burst repetition rate, each burst being separated by a predetermined rest period.

25. The apparatus of claim 21 where the source of the LSEFN electric field comprises a source which generates an LSEFN electric field of less than 50 v/cm.

26. The apparatus of claim 21 where the source of the LSEFN electric field comprises a source which generates an LSEFN electric field of approximately 10 v/cm or less.

27. The apparatus of claim 21 where the source of the LSEFN electric field comprises a source which generates an LSEFN electric field of approximately 1 v/cm or less.

28. The apparatus of claim 21 where the source of the LSEFN electric field comprises a source which generates an LSEFN electric field of less than a determined value which causes dielectric heating and biological damage to the cells, cell clusters, or tissues.

29. The apparatus of claim 21 where the source of the LSEFN electric field comprises at least one pair of electrodes across which the electric field is imposed, the electrodes being arranged and configured to provide a fringing field between them and being separated by a distance such that the cells, cell clusters, or tissues are primarily exposed to the fringing field so that the cells, cell clusters, or tissues are exposed to the averaged field strength and the averaged electrical polarization of the LSEFN electric field.

30. The apparatus of claim 21 where the source of the LSEFN electric field comprises an electrode array, and a chamber with walls in or on which the electrode array is disposed which generate the LSEFN electric field and which walls intimately conform to the cells, cell clusters, or tissues subject to LSEFN, thereby providing the averaged field strength and the averaged electrical polarization of the LSEFN electric field.

31. The apparatus of claim 22 where the bath provides the flowing fluid to move the cells, cell clusters, or tissues in the LSEFN electric field.

32. The apparatus of claim 31 where the bath provides the flowing fluid to rotate the cells, cell clusters, or tissues in the LSEFN electric field.

33. The apparatus of claim 32 where the bath provides the flowing fluid to tumble the cells, cell clusters, or tissues in the LSEFN electric field.

34. The apparatus of claim 22 where the bath provides the flowing fluid to maintain a temperature of the fluid substantially constant to avoid heat damage to the cells, cell clusters, or tissues in the LSEFN electric field.

35. The apparatus of claim 21 where the source of LSEFN electric field comprises a multiple arrays of a plurality of small electrodes to generate a pixilated fringing electric field.

36. The apparatus of claim 21 where the source of LSEFN electric field and the systemic transfusing source are arranged and configured to accommodate a large number of cells, cell clusters, or tissues in a batch during a single exposure time interval over an extended exposure path along which the cells, cell clusters, or tissues are moved, whereby mass production of mediated cells, cell clusters, or tissues are produced.

37. The apparatus of claim 36 where the sources are folded into a compact volume while providing the extended exposure path.

38. An apparatus of delivery of gene, protein or drug materials to macroscopic quantities of cells, cell clusters, or tissues comprising:
a source of a dynamic ultra low strength electric field electroporation for ex vivo exposure to the cells, cell clusters, or tissues;
a means for systemically transfusing the gene, protein or drug materials into the cells, cell clusters, or tissues during LSEFN; and
a gas permeable tissue culture chamber, which intimately conformed to the cells, cell clusters or tissues; and
a microscope for determining the systemic transfusing of the gene, protein or drug materials into the cells, cell clusters, or tissues during LSEFN.

39. The apparatus of claim 38 where source of a dynamic ultra low strength electric field electroporation comprises a generator of low electric field LSEFN pulses; and an array of opposing electrodes coupled to the generator; and
where the gas permeable tissue culture chamber comprises a chamber having walls formed by membranes enclosing the array of opposing electrodes defining an LSEFN and transfusion chamber defined between the opposing electrodes of the array, which chamber is shaped and sized to intimately contain the cells, cell clusters, or tissues between opposing membrane encapsulated electrode arrays across which LSEFN pulses are applied while gene, protein or drug materials are flowed through the chamber for a predetermined time; and
further comprising a closed, sterile and temperature controlled circulating culture buffer perfusion system communicated to the chamber, which keeps the cells and cell clusters rolling in the electric field and receiving homogenously distributed LSEFN while the cell, cell clusters and tissue maintained in a sterile and nutritional cell and tissue culture environment.
